(19) United States
(12) Patent Application Publication
(10) Pub. No.: US 2025/0140392 A1
(43) Pub. Date: May 1, 2025

(54) METHOD FOR RETRIEVING INFORMATION ABOUT SIMILAR MEDICAL DEVICES AND COMPUTER DEVICE USING THE SAME

(71) Applicant: NATIONAL APPLIED RESEARCH LABORATORIES, TAIPEI CITY (TW)

(72) Inventors: BOU-WEN LIN, TAIPEI CITY (TW); KUAN-JU WANG, TAIPEI CITY (TW); CHIH-YU AN, TAIPEI CITY (TW)

(21) Appl. No.: 18/498,061

(22) Filed: Oct. 31, 2023

(51) Int. Cl. G16H 40/40; G16H 50/20

(52) U.S. Cl. CPC G16H 40/40; G16H 50/20

(57) ABSTRACT

A method for retrieving information about similar medical devices is provided, which includes the following steps: obtaining a first technical context of a specific medical device; utilizing a first machine-learning model to extract one or more technical items of the specific medical device based on technical content of the first technical context; utilizing the first machine-learning model to generate candidate medical devices using the technical items; searching a database for device information about the candidate medical devices; retrieving summary files of the candidate medical devices from the database based on the device information; utilizing the first machine-learning model to infer a second technical context of each candidate medical device; and determining a most similar medical device for the specific medical device according to a similarity score for each candidate medical device calculated from the second technical context and the first technical context using a second machine-learning model.

### FIELD OF THE INVENTION

The present disclosure relates to computer devices, and, in particular, a method for retrieving information about similar medical devices and a computer device using the same.

### BACKGROUND

After the completion of research and development, medical devices must undergo certification through regulatory channels before they can be marketed and sold. During the review process for regulatory certification, manufacturers are required to align their developed medical devices with existing predicate medical devices (i.e., legally marketed medical devices) in order to expedite the review process. However, manufacturers often face challenges in discovering suitable or similar predicate medical devices due to a disparity between professional knowledge and the current medical device classification architecture.

### SUMMARY OF THE INVENTION

In an aspect of the present disclosure, a method for retrieving information about similar medical devices is provided. The method includes the following steps: obtaining a first technical context of a specific medical device; utilizing a first machine-learning model to extract one or more technical items of the specific medical device based on technical content of the first technical context; utilizing the first machine-learning model to generate one or more candidate medical devices using the one or more technical items of the specific medical device; searching a database for respective device information about each candidate medical device; retrieving a summary file of each candidate medical device from the database based on the respective device information of each candidate medical device; determining a most similar medical device for the specific medical device according to a similarity score for each candidate medical device calculated from the second technical context of each candidate medical device and the first technical context of the specific medical device using a second machine-learning model.

In another aspect of the present disclosure, a computer device for retrieving information about similar medical devices is provided. The computer device includes a memory and a processor. The memory has computer executable instructions stored therein. The processor is coupled to the memory. The computer executable instructions cause the processor to perform operations, and the operations include: obtaining a first technical context of a specific medical device; utilizing a first machine-learning model to extract one or more technical items of the specific medical device based on technical content of the first technical context; utilizing the first machine-learning model to generate one or more candidate medical devices using the one or more technical items of the specific medical device; searching a database for respective device information about each candidate medical devices; retrieving a summary file of each candidate medical device from the database based on the device information of the one or more candidate medical devices; utilizing the first machine-learning model to infer a second technical context of each candidate medical device from the summary file or each candidate medical device; and determining a most similar medical device for the specific medical device according to a similarity score for each candidate medical device calculated from the second technical context of each candidate medical device and the first technical context of the specific medical device using a second machine-learning model.

### BRIEF DESCRIPTION OF THE DRAWINGS

Aspects of the present disclosure are best understood from the following detailed description when read with the accompanying figures. It is noted that in accordance with the standard practice in the industry, various features are not drawn to scale. In fact, the dimensions of the various features may be arbitrarily increased or reduced for clarity of discussion.

FIG. 1 is a block diagram of a computer system in accordance with an embodiment of the present disclosure.

FIG. 2 is a flowchart of a method for retrieving information about similar medical devices in accordance with an embodiment of the present disclosure.

FIG. 3 is a flowchart of step 220 in accordance with the embodiment of in FIG. 2.

FIG. 4 is a flowchart of step 270 in accordance with the embodiment of in FIG. 2.

Corresponding numerals and symbols in the different figures generally refer to corresponding parts unless otherwise indicated. The figures are drawn to clearly illustrate the relevant aspects of the various embodiments and are not necessarily drawn to scale.

### DETAILED DESCRIPTION

The following disclosure provides many different embodiments, or examples, for implementing different features of the provided subject matter. Specific examples of operations, components, and arrangements are described below to simplify the present disclosure. These are, of course, merely examples and are not intended to be limiting. For example, a first operation performed before or after a second operation in the description may include embodiments in which the first and second operations are performed together, and may also include embodiments in which additional operations may be performed between the first and second operations. For example, the formation of a first feature over, on or in a second feature in the description that follows may include embodiments in which the first and second features are formed in direct contact, and may also include embodiments in which additional features may be formed between the first and second features, such that the first and second features may not be in direct contact. In addition, the present disclosure may repeat reference numerals and/or letters in the various examples. This repetition is for the purpose of simplicity and clarity and does not in itself dictate a relationship between the various embodiments and/or configurations discussed.

Time relative terms, such as “prior to,” “before,” “posterior to,” “after” and the like, may be used herein for ease of description to describe one operations or feature's relationship to another operation(s) or feature(s) as illustrated in the figures. The time relative terms are intended to encompass different sequences of the operations depicted in the figures. Further, spatially relative terms, such as “beneath,” “below,” “lower,” “above,” “upper” and the like, may be used herein for ease of description to describe one element or feature's relationship to another element(s) or feature(s) as illustrated in the figures. The spatially relative terms are intended to encompass different orientations of the device in use or operation in addition to the orientation depicted in the figures. The apparatus may be otherwise oriented (rotated 90 degrees or at other orientations) and the spatially relative descriptors used herein may likewise be interpreted accordingly. Relative terms for connections, such as “connect,” “connected,” “connection,” “couple,” “coupled,” “in communication,” and the like, may be used herein for ease of description to describe an operational connection, coupling, or linking one between two elements or features. The relative terms for connections are intended to encompass different connections, coupling, or linking of the devices or components. The devices or components may be directly or indirectly connected, coupled, or linked to one another through, for example, another set of components. The devices or components may be wired and/or wireless connected, coupled, or linked with each other.

As used herein, the singular terms “a,” “an,” and “the” may include plural referents unless the technical context clearly indicates otherwise. For example, reference to a device may include multiple devices unless the technical context clearly indicates otherwise. The terms “comprising” and “including” may indicate the existences of the described features, integers, steps, operations, elements, and/or components, but may not exclude the existences of combinations of one or more of the features, integers, steps, operations, elements, and/or components. The term “and/or” may include any or all combinations of one or more listed items.

Additionally, amounts, ratios, and other numerical values are sometimes presented herein in a range format. It is to be understood that such range format is used for convenience and brevity and should be understood flexibly to include numerical values explicitly specified as limits of a range, but also to include all individual numerical values or sub-ranges encompassed within that range as if each numerical value and sub-range is explicitly specified.

The nature and use of the embodiments are discussed in detail as follows. It should be appreciated, however, that the present disclosure provides many applicable inventive concepts that can be embodied in a wide variety of specific contexts. The specific embodiments discussed are merely illustrative of specific ways to embody and use the disclosure, without limiting the scope thereof.

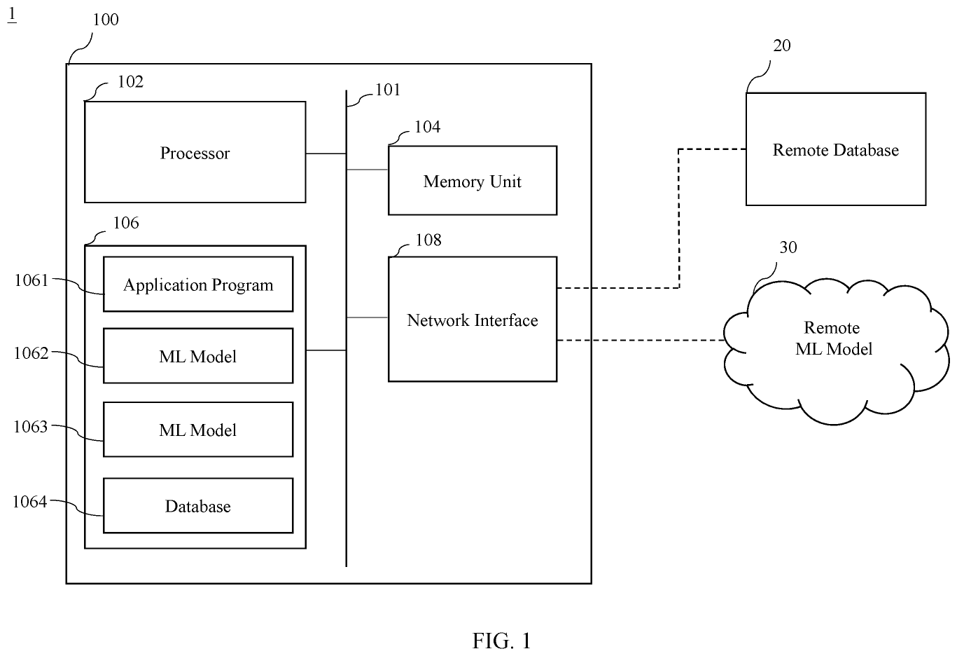

FIG. 1 is a block diagram of a computer system in accordance with an embodiment of the present disclosure.

In some embodiments, the computer system 1 may include a computer device 100, a remote database 20, and a remote machine-learning (ML) model 30. The computer device 100 may comprise, but not limited to, mobile phones, desktop computers, laptops, personal digital assistants (PDAs), smartphones, tablets, laptops, multi-processor systems, microprocessor-based or programmable consumer electronics, game consoles, set-top boxes, or any other suitable devices with computing and network capabilities. The computer device 100 may include a processor 102, a memory unit 104, a storage device 106, and a network interface 108 that are electrically connected to each other through bus 101, as depicted in FIG. 1.

In some embodiments, the processor 102 may be or include one or more central processor units (CPUs), microprocessors, co-processing entities, field programmable gate array (FPGAs), application-specific integrated circuits (ASICs), or any other circuitry having processing capability, but the present disclosure is not limited thereto. The memory unit 104 may be a volatile memory such as a dynamic random access memory (DRAM) or a static random access memory (SRAM) which serves as an execute space and stores intermediate data for an application program 1061.

In some embodiments, the storage device 106 may be a non-volatile memory such as a hard disk drive (HDD), a flash memory, a read-only memory, SD memory card, memory sticks, ferroelectric random access memory (FeRAM), resistive random access memory (RRAM), etc., but the present disclosure is not limited thereto. In some embodiments, the storage device 106 stores the application program 1061, machine-learning models 1062 and 1063, and a database 1064. The application program 1061 may include instructions to be executed by the processor 102 perform operations for retrieving information about similar medical devices from a remote database 20, as will further explained. The machine-learning model 1062 may be configured to remove adverbs, punctuations, stopwords, and other unnecessary elements (e.g., accent marks, diacritics, etc.) from a context. The machine-learning model 1063 may be configured to deduce an abstract from a technical context, as will further explained. The database 1064 may store information pertaining to manufacturers and their products, as well as potential application domains in the field of medical device manufacturing.

In some embodiments, the network interface 108 supports wired and/or wireless transmission protocols that enable communication with remote database 20 and remote machine-learning model 30. The wired transmission protocols may include Ethernet, Universal Serial Bus (USB), Inter Integrated Circuit (I2C), Serial Peripheral Interface (SPI), etc., but the present disclosure is not limited thereto. The wireless transmission protocols may include Wi-Fi (802.11), Global System for Mobile Communications (GSM), Enhanced Data rates for GSM Evolution (EDGE), Time Division-Synchronous Code Division Multiple Access (TD-SCDMA), Long Term Evolution (LTE), 4-th Generation (4G), 5-th Generation (5G), 6-th Generation (6G), ultra-wideband (UWB), infrared (IR) protocols, near field communication (NFC) protocols, Wibree, Bluetooth protocols, wireless universal serial bus (USB) protocols, but the present disclosure is not limited thereto.

In some alternative embodiments, the remote database 20 may be a public database (e.g., OpenFDA) initiated by the U.S. Food and Drug Administration (FDA) to provide public access to a vast amount of data related to approved drugs, medical devices, and food products. OpenFDA offers a user-friendly interface and API (Application Programming Interface) that allows users to search and retrieve data about the approved drugs, medical devices, and food products. The remote machine-learning model 30 can be a pre-existing, large generative pre-trained transformer (GPT) model sourced from online platforms.

Taking the Food and Drug Administration (FDA) in the United States as an example, the approval procedure for a Class II medical device may involve the following steps: 1) Device Classification; 2) Pre-market Notification (e.g., 510(k)); 3) Preparing the 510(k) submission; 4) FDA review; 5) FDA decision; and 6) Post-Market Surveillance. It should be noted that the approval procedure can vary depending on the specific medical device and its intended use.

The FDA categorizes medical devices into three classes (Class I, II, and III) based on their risk level. Class II medical devices are considered to have moderate risk. Manufacturers of Class I and Class II medical devices need to submit a pre-market notification (e.g., 510(k)) to demonstrate that the medical device is substantially equivalent to a legally marketed medical device (i.e., predicate device) that does not require pre-market approval. For example, the submission may include details information about the device, its intended use, performance data, labeling, and any applicable clinical studies or testing. The FDA will review the 510(k) submission to assess the medical device's safety and effectiveness and to determine whether the medical device is substantially equivalent to the predicate device by evaluating the medical device's intended use, design, materials, performance, and labeling. If the FDA determines that the medical device is substantially equivalent to the predicate device, it can be marketed and sold in the U.S, which takes shorter time for FDA approval. If the FDA determines that the medical device is not substantially equivalent to the predicate device, the manufacturer may need to pursue other regulatory pathways, such as a pre-market approval (PMA) application, which takes much longer time for FDA approval.

Specifically, when developing a new medical device, manufacturers often conduct a comprehensive search for similar medical devices that have received approval from regulatory authorities such as the FDA. This search may be conducted through online platforms, as well as public or private databases, which yield a variety of potential candidates. Subsequently, the manufacturer analyzes the technical characteristics of these candidates to identify the most similar medical devices, which aids in preparing the submission for regulatory approval. However, the new medical device may possess some unique technical features, effects, and advantages that differ from any predicate medical devices in the same category. Consequently, the regulatory authority may determine that the new medical device is not substantially equivalent to any predicate devices included in the submission. Additionally, there may be instances where the most similar predicate medical device belongs to a different category, making it challenging to obtain information about similar medical devices.

In an embodiment, upon the application program 1061 being executed by the processor 102, the processor 102 may obtain a technical context describing a specific medical device. The specific medical device can be a newly developed medical device or one that has not yet received approval. The technical context may be in the form of an abstract or technical description that provides information about the specific medical device, including its intended use, technical characteristics, technical improvements, advantages, etc.

In an embodiment, the specific medical device is an ultrasound-imaging guided robotic HIFU ablation system for tumor treatment with a respiration and displacement compensatory mechanism. An example of such a technical context for the specific medical device is presented below: “In recent years, high-intensity focused ultrasound (HIFU) has commonly been applied in non-invasive tumor therapy, such as to treat uterine fibroids or prostate tumors. However, respiration may cause tumor displacement such as liver tumor, which may lead to error in localization or inadequate thermal effects on the tumor. The goal of this research is to develop an ultrasound-imaging guided robotic HIFU ablation system for tumor treatment with a respiration and displacement compensatory mechanism. The system integrates the technologies of ultrasound image assisted guidance, robotic positioning control, and HIFU treatment planning. With the assistance of ultrasound image guidance technology, the tumor size and location can be determined from ultrasound images as well as the robotic arm can be controlled to position the HIFU transducer to focus on the target tumor. According to the correlation between the measured displacements of the heaving chest and the target tumor, a respiration simulation device was designed, which used a phantom and a cam-driving mechanism to simulate displacements of the tumor and of the heaving chest. Then, a polynomial function of tumor position relative to the position of the heaving chest was generated. After the coordinate frames of the robotic arm, optic tracker and tumor phantom had been registered, the robotic arm was able to guide the HIFU probe to track and ablate the target tumor automatically and synchronously by inputting the displacement values of the heaving chest.”

After obtaining the aforementioned context, the processor 102 may proceed to execute the machine-learning model 1062 in order to perform a text cleaning process. The text cleaning process involves removing adverbs, punctuations (such as periods, commas, question marks), stopwords, and other unnecessary elements from the technical context, which may be in English, traditional Chinese, simplified Chinese, or other language, so primary technical content and/or keywords will be retained in the raw text of the cleaned context. The machine-learning model 1062 used in this process may be a natural language processing (NLP) model that can identify relationships between the various elements of language, such as the letters, words, phrases, and sentences present within in the technical context. Additionally, the machine-learning model 1062 is capable of identifying and removing adverbs, punctuations, stopwords, and other unwanted elements from the technical context, thereby facilitating the text cleaning process.

After the completion of the text cleaning process, the processor 102 may send the primary technical content and/or keywords in the cleaned technical context to the remote machine-learning model 30 to generate technical items of the cleaned technical context of the specific medical device. Each of the technical items may include a possible application domain and its technical description that are either explicitly mentioned in the cleaned technical context or inferred by the remote machine-learning model 30. The technical description of the potential application domain in each technical item may include advantages of the specific medical device and/or physical systems, components, or equipment of the specific medical device. Specifically, the primary content and/or keywords within the cleaned technical context utilized by the remote machine-learning model 30 can yield more precise inferred technical items compared to the original technical context of the specific medical device.

An example of technical items and their technical analysis and potential application domain for the specific medical device is presented below:
1) Non-invasive treatment of liver tumors: The ultrasound-imaging guided robotic HIFU ablation system can compensate for tumor displacement caused by respiration, ensuring accurate localization and effective thermal effects on liver tumors.
2) Treatment of other types of tumors: The system can be adapted to treat various types of tumors, such as breast tumors or kidney tumors, by adjusting the ultrasound image guidance and robotic positioning control.
3) Improved precision in tumor ablation: The integration of ultrasound image assisted guidance, robotic positioning control, and HIFU treatment planning allows for precise targeting and ablation of tumors, minimizing damage to surrounding healthy tissues.
4) Enhanced safety in tumor treatment: By automatically tracking and ablating the target tumor, the system reduces the risk of human error and improves the safety of the procedure.
5) Potential for outpatient tumor treatment: The non-invasive nature of the system and its ability to accurately target tumors may make it suitable for outpatient tumor treatment, reducing the need for hospitalization.
6) Minimally invasive treatment of uterine fibroids: Building on the existing application of HIFU in uterine fibroid treatment, the system can further improve the precision and effectiveness of the procedure.
7) Treatment of recurrent tumors: The system's ability to accurately track and ablate tumors makes it a potential option for treating recurrent tumors that may have shifted in position.
8) Potential for combination therapy: The ultrasound-imaging guided robotic HIFU ablation system can be combined with other treatment modalities, such as chemotherapy or radiation therapy, to enhance the overall effectiveness of tumor treatment.
9) Research tool for studying tumor behavior: The system can be used as a research tool to study the behavior of tumors under different conditions, such as tumor displacement caused by respiration, providing valuable insights for future treatment strategies.
10) Potential for personalized tumor treatment: The system's ability to accurately localize and target tumors opens up possibilities for personalized treatment approaches, tailoring the treatment to the specific characteristics of each patient's tumor.

In some embodiments, the processor 102 may initiate a search for manufacturers and their corresponding medical devices that are relevant to the technical items, including their potential application domains and corresponding technical description. This search can be conducted using either the database 1064 or the remote machine-learning model 30. In some embodiments, the database 1064 is a pre-existing database established by the manufacturer of the specific medical device or by database companies. The database 1064 contains information on predicate medical devices and their respective technical features. The processor 102 may first search the database 1064 for manufacturers associated with the potential application domains of the specific medical device from the database 1064, based on the potential application domains of the technical items obtained from the cleaned technical context of the specific medical device. Alternatively, the processor 102 may utilize the remote machine-learning model 30 to generate manufacturers associated with the potential application domains of the specific medical device based on the potential application domains of the technical items obtained from the cleaned technical context of the specific medical device.

In some embodiments, the potential application domain of each technical item of the specific medical device obtained from the database 1064 or inferred by the remote machine-learning model 30 may be the application domain of the specific medical device itself or a broader concept. Further details will be provided to illustrate some examples of these potential application domains.

The first technical item mentions the potential application domain of “Non-invasive treatment of liver tumors,” which refers to the desired application domain of the specific medical device. The second technical item introduces the potential application domain of “Treatment of other types of tumors,” which represents a broader concept beyond the original application domain of the specific medical device. This means that the specific medical device can potentially be used for treating various types of tumors, such as breast tumors or kidney tumors. The third technical item highlights the potential application domain of “Improved precision in tumor ablation,” which also falls under a broader concept. This is because the specific medical device's accuracy is emphasized in the technical context, suggesting that it may lead to the development of other robotic-related surgical systems. Similarly, the fourth technical item mentions the potential application domain of “Enhanced safety in tumor treatment,” which is also a broader concept. This technical item is related to image-guided surgical treatments, such as endoscopic surgery, computer-tomography (CT) imaging-guided surgery, or magnetic resonance imaging (MRI) guided surgery. Lastly, the eighth technical item discusses the potential application domain of “Potential for combination therapy,” which is another broader concept. In this case, ultrasound images can be utilized to guide other types of surgery, such as irradiation therapy or targeted therapy.

In some embodiments, the processor 102 can send the technical items, including potential application domains and their technical description for the specific medical device, to the remote machine-learning model 30. The remote machine-learning model 30 can generate one or more candidate medical devices and their respective manufacturers based on the potential application domains and their technical description of the specific medical device.

More specifically, the process of searching for manufacturers and their products extends beyond the original application domains and medical-device classification of the specific medical device. It encompasses a broader concept, allowing for increased flexibility and efficiency in retrieving information about similar medical devices for the specific medical device.

In some embodiments, these manufacturers may be considered benchmark companies in the field of medical device manufacturing. An example of such benchmark manufacturers associated with the potential application domains of the specific medical device is presented below:

### [Benchmark Company]

1. SonaCare Medical LLC
2. EDAP TMS S.A.
3. Alpinion Medical Systems Co., Ltd.
4. Profound Medical Corp.
5. Philips Healthcare

Subsequently, the processor 102 may search the database 1064 for products (e.g., Class II medical devices) manufactured by these manufacturers that are similar to the specific medical device, based on the technical items and their technical analysis of the specific medical device.

In some embodiments, these medical devices are considered the benchmark products of each manufacturer. An example of such benchmark product associated with the technical items and their technical analysis of the specific medical device is presented below:

### [Benchmark Product]

1. SonaCare Medical LLC, Sonablate
2. EDAP TMS S.A., Ablatherm
3. Alpinion Medical Systems Co., Ltd., E-CUBE series
4. Profound Medical Corp., TULSA-PRO
5. Philips, EPIQ Elite Ultrasound System

Upon discovering the products, such as medical devices, manufactured by various manufacturers, the processor is capable of generating a search list. The processor 102 can further organize each medical device and its corresponding manufacturer on the search list in a specific search string that conforms to the input syntax of the API of the remote database 20, such as OpenFDA.

For instance, the specific search string for the medical device “Ablatherm” (i.e., device name) manufactured by “EDAP TMS S.A.” (i.e., manufacturer's name) would be as follows: https://api.fda.gov/device/510k.json?search=(applicant: “EDAP”+OR+applicant: “TMS”+OR+applicant: “S.A.”)+AND+ (device_name: “Ablatherm”) &limit=10, where 10 represents the maximum numbers of medical devices reported by the OpenFDA per search, and it can be adjusted as needed. Specifically, any word from the manufacturer's name (e.g., EDAP, TMS, or S.A) and any word from the candidate medical device's name (e.g., Ablatherm) can be regarded as a combination indicated by the search string to query the remote database 20. In this example, three different combinations, such as Applicant=EDAP & device_name=Ablatherm, Applicant=TMS & device_name=Ablatherm, and Applicant=S.A. & device_name=Ablatherm, will be searched in the remote database 20. In some embodiments, some specific words or abbreviations, such as “LLC”, “Corp.”, “Inc.”, “Co.”, “Ltd.”, etc., in the manufacturer's name may be omitted while generating the search string for the remote database 20.

Specifically, the processor 102 will utilize the aforementioned specific string to search the OpenFDA database for the Applicant “EDAP TMS S.A.” and the medical device “Ablatherm”. Similar search procedures will be conducted for the other medical devices on the search list in the OpenFDA database. It should be noted that the API of the OpenFDA database will provide the search results for each specific string corresponding to the medical devices on the list in a designated data file, typically in JSON (JavaScript Object Notation) format. JSON is a lightweight data-interchange format that is easily readable and writable by humans, as well as easily parsed and generated by machines. It is commonly used for transmitting data between servers and web applications, serving as an alternative to XML. JSON files are text files with a .json extension and consist of key-value pairs, where the keys are strings and the values can be strings, numbers, booleans, arrays, or other JSON objects. JSON is widely supported by programming languages and is a popular format for storing and exchanging data.

After obtaining the JSON files, the processor 102 may parse the JSON files associated with the medical devices on the search list obtained from the OpenFDA in order to retrieve information about candidate medical devices similar to the specific medical device. The information includes device class, 510(k) number, applicant, device name, decision date, 510(k) submission file (e.g., in PDF format), etc. It should be noted that the JSON file may contain multiple candidate medical devices per search, so for the sake of brevity, a simplified list of the retrieved information about the candidate medical devices similar to the specific medical device is provided below:

### [SonaCare Medical LLC, Sonablate]:

### DeviceClass: 2, 510(k) Number: K160942, Applicant: SonaCare Medical, LLC, DeviceName: Sonablate, DecisionDate: 2016 Dec. 21,

### [EDAP TMS S.A., Ablatherm]:

### DeviceClass: 2, 510(k) Number: K172285, Applicant: EDAP TECHNOMED, INC., DeviceName: Ablatherm Fusion, DecisionDate: 2017 Oct. 3,

### [Alpinion Medical Systems Co., Ltd., E-CUBE Series]:

DeviceClass: 2, 510(k) Number: K230925, Applicant: Alton (Shanghai) Medical Instruments Co. Ltd, DeviceName: Disposable Injection Needle AF series, DecisionDate: 2023 Sep. 27,

### [Profound Medical Corp., TULSA-PRO]:

### DeviceClass: 2, 510(k) Number: K230692, Applicant: Profound Medical Inc., DeviceName: TULSA-PRO System, DecisionDate: 2023 Sep. 20,

### [Philips Healthcare, Philips EPIQ Elite Ultrasound System]:

### DeviceClass: 2, 510(k) Number: K231190, Applicant: Philips Ultrasound LLC, DeviceName: EPIQ Series Diagnostic Ultrasound System, DecisionDate: 2023 May 12.

In some embodiments, the processor 102 adds a hyperlink to the 510(k) number of each candidate medical device listed above, allowing users to easily access the respective 510 (k) submission file (in PDF format) from the OpenFDA. It should be noted that the JSON file provided by the OpenFDA does not contain the summary files or hyperlinks for the candidate medical devices on the list. However, the processor 102 is capable of identifying the 510 (k) numbers in the JSON file and appending the appropriate hyperlinks to the corresponding candidate medical devices in the list.

In some embodiments, the processor 102 retrieves the summary files of the candidate medical devices by utilizing their respective 510(k) numbers on the list. The content within these summary files is then parsed to extract technical information, such as technical descriptions (e.g., device description, intended use, indications for use, summary of technological characteristics, etc.) or specifications, pertaining to the candidate medical devices. For instance, the medical device “Ablatherm” manufactured by EDAP TMS S.A. has a 510(k) number of K172285. The processor 102 can directly access the summary file of this particular medical device using the defined syntax of the OpenFDA's API. In this case, the hyperlink for accessing the summary file of the medical device “Ablatherm” is https://www.accessdata.fda.gov/scripts/cdrh/cfdocs/cfpmn/pmn.cfm?ID=K1722 85. In some embodiments, the list of the candidate medical devices can be presented to the users, allowing the users to assess whether these candidate medical devices align with their specific needs and requirements.

In some embodiments, the processor 102 may utilize the remote machine-learning model 30 to infer an abstract (e.g., another technical context) of each candidate medical device on the list based on the summary file of each candidate medical device. Subsequently, the processor 102 may utilize the machine-learning model 1063 to convert the technical items of the specific medical device and the abstract of the technical context of each candidate medical device into a first vector group and a second vector group, respectively, using the technique of “word to vector” (e.g., Word2vec). For example, the machine-learning model 1063 could be a pre-trained natural language processing model capable of converting the specific technical elements of the individual medical device and the abstract of the technical context of each potential medical device into distinct vector groups using the “word to vector” technique, such as Word2vec. Word2vec is a two-layer neural network that processes text by transforming words into feature vectors, thereby representing them within a given corpus.

After the first vector group and the second vector group are obtained, the processor 102 may calculate similarities (e.g., cosine similarities) between the vectors in the first vector group and those in the second vector group to determine a similarity score of each candidate medical device. For example, if there are N vectors denoted as A1 to AN in the first vector group, and there are M vectors denoted as B1 to BM in the second vector group, the first vector group and the second vector group can be regarded as an N-dimension vector A and a M-dimension vector B, respectively, where M and N are positive integers. If N is greater than M, the processor 102 may append zeros to the vector B (e.g., a zero padding process), resulting in the padded vector B with a dimension equal to N. In such case, the cosine similarity SC between the first vector group (e.g., N-dimension vector A) and the second vector group (e.g., N-dimension padded vector B) can be calculated using the formula (1) as follows:

$$S C ( A , B ) = cos ( θ ) = \frac{A \cdot B}{‖ A ‖ ‖ B ‖} = \frac{{\sum }_{i = 1}^{N} {A}_{i} {B}_{i}}{\sqrt{{\sum }_{i = 1}^{N} {A}_{i}^{2}} \cdot \sqrt{{\sum }_{i = 1}^{N} {B}_{i}^{2}}} \qquad ( 1 )$$

If N is less than M, the processor 102 may append zeros to the vector A (e.g., a zero padding process), resulting in the padded vector A with a dimension equal to M. In such case, the cosine similarity SC between the first vector group (e.g., M-dimension padded vector A) and the second vector group (e.g., M-dimension vector B) can be calculated using the formula (2) as follows:

$$S C ( A , B ) = cos ( θ ) = \frac{A \cdot B}{‖ A ‖ ‖ B ‖} = \frac{{\sum }_{i = 1}^{M} {A}_{i} {B}_{i}}{\sqrt{{\sum }_{i = 1}^{M} {A}_{i}^{2}} \cdot \sqrt{{\sum }_{i = 1}^{M} {B}_{i}^{2}}} \qquad ( 2 )$$

Cosine similarity is a measure of similarity between two non-zero vectors defined in an inner product space. Cosine similarity is the cosine of the inner angle between the vectors; that is, it is the dot product of the vectors divided by the product of their lengths. The cosine similarity always belongs to the interval [−1, 1]. For instance, when the inner angle between two vectors approaches 0 degrees, the cosine similarity tends towards 1, indicating a high level of similarity. If the inner angle approaches 180 degrees, the cosine similarity approaches −1, suggesting a lack of similarity. When the inner angle between two vectors is 90 degrees, the cosine similarity is 0, indicating no similarity between the vectors.

The calculated cosine similarity between the abstract of a particular candidate medical device and the technical items of the specific medical device can be used as the similarity score of the particular candidate medical device. The similarity scores of other candidate medical devices can be calculated in a similar manner. For example, each candidate medical device on the list may have its own respective similarity score. A higher similarity score of a particular candidate medical device indicates a higher similarity to the specific medical device. The processor 102 will identify that candidate medical device with the highest similarity score as the most similar medical device to the specific medical device. Since machine-learning models and natural language processing techniques are employed to convert the technical context (e.g., an abstract) of each candidate medical device into mathematical vectors. This enables the calculation of similarities between candidate medical devices and the specific medical device in a more intelligent manner, resulting in time and energy savings for the users.

Since the 510(k) number of the most similar medical device is already known from the list, the processor 102 can retrieve the technical specification of this medical device from its summary file. Consequently, the retrieved technical specification can be provided to the user, thereby assisting the user to prepare the 510(k) submission for the specific medical device. For example, the feature comparison chart between features of the most similar medical device and the specific medical device can provide evidence that the specific medical device is substantially equivalent to the most similar medical device (e.g., a predicate device), by comparing their features.

In view of the above, the technique described in the aforementioned embodiments is capable of automatically retrieving information about similar medical devices based on a technical context of the specific medical device. As a result, this approach reduces the complexity and time required for the search process. The search for similar medical devices is not restricted to medical devices within the same category as the specific medical device, but rather utilizes potential application domains and corresponding technical description of the technical items derived from the technical context of the specific medical target device. This bridges the gap between professional knowledge and the existing medical-device classification architecture, facilitating a better understanding of the medical professional field for users. Additionally, the data of candidate medical devices undergo multiple processing steps, thereby enhancing the accuracy of the search results. Therefore, the technique described in the present disclosure offers a more efficient and convenient way for professionals in the medical device field to retrieve information about similar medical devices with higher levels of accuracy.

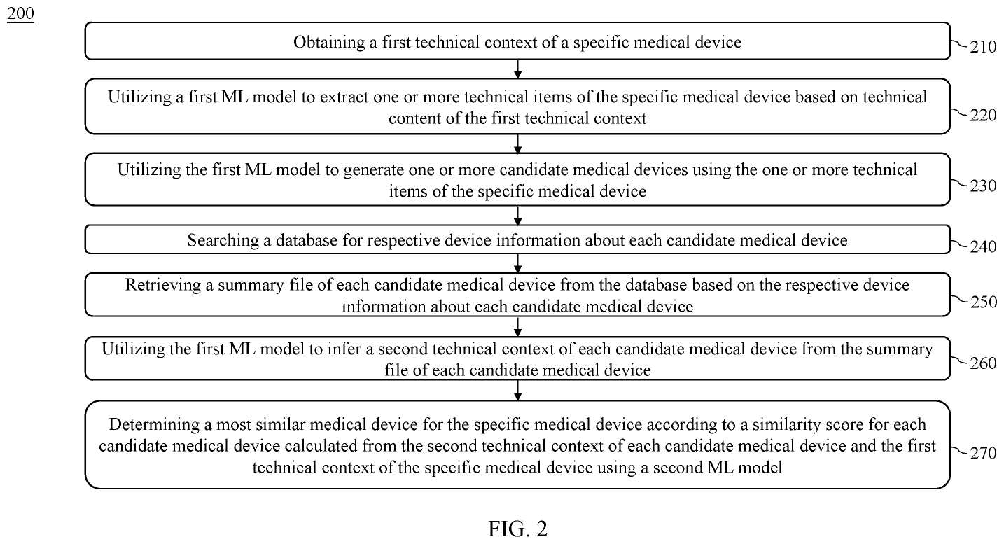

FIG. 2 is a flowchart of a method for retrieving information about similar medical devices in accordance with an embodiment of the present disclosure. Please refer to FIG. 1 and FIG. 2.

In an embodiment, the method 200 for retrieving information about similar medical devices may include the following steps: Step 210: Obtaining a first technical context of a specific medical device. For example, the specific medical device can be a newly developed medical device or one that has not yet received approval. The technical context may be in the form of an abstract or technical description that provides information about the specific medical device, including its intended use, technical characteristics, technical improvements, advantages, etc.

Step 220: Utilizing a first machine-learning model to extract one or more technical items of the specific medical device based on technical content and/or keywords in the technical context. For example, the first machine-learning model may be the remote machine-learning model 30. Once the technical context is obtained, the processor 102 may execute the machine-learning model 1062 to perform a text cleaning process. The text cleaning process involves removing adverbs, punctuations (such as periods, commas, question marks), stopwords, and other unnecessary elements from the technical context. By doing so, the primary technical content and/or keywords are preserved in the raw text of the cleaned context. The primary content and/or keywords within the cleaned technical context utilized by the remote machine-learning model 30 can yield more precise inferred technical items, accompanied with their respective technical analysis and potential application domains, in comparison to the original technical context of the specific medical device.

Step 230: Utilizing the first machine-learning model to generate one or more candidate medical devices using the one or more technical items of the specific medical device. For example, the processor 102 can send the technical items, including respective potential application domains and corresponding technical description for the specific medical device, to the remote machine-learning model 30. The remote machine-learning model 30 can generate one or more candidate medical devices and their manufacturers based on the technical items and their technical analysis and potential application domains of the specific medical device.

Step 240: Searching a database for respective device information about each candidate medical device. For example, the database (e.g., remote database 20) may be a public database containing device information of a vast amount of predicate medical devices, such as OpenFDA initiated by the U.S. Food and Drug Administration (FDA). The device information about each of the candidate medical devices may include a device Class, 510(k) number (e.g., a submission number), applicant, device name, decision date, etc.

Step 250: Retrieving a summary file of each candidate medical device from the database based on the respective device information of each candidate medical device. For example, the processor 102 may retrieve the summary files of the candidate medical devices by utilizing their respective 510(k) numbers in a defined syntax of the OpenFDA's API. The content within these summary files is then parsed to extract technical information, such as technical descriptions (e.g., device description, intended use, indications for use, summary of technological characteristics, etc.) or specifications, pertaining to the candidate medical devices.

Step 260: Utilizing the first machine-learning model to infer a second technical context of each candidate medical device from the summary file or each candidate medical device. For example, the second technical context may be an abstract of each candidate medical device. The remote machine-learning model 30 can infer an abstract of each candidate medical device based on technical descriptions or specification of the summary file of each candidate medical device.

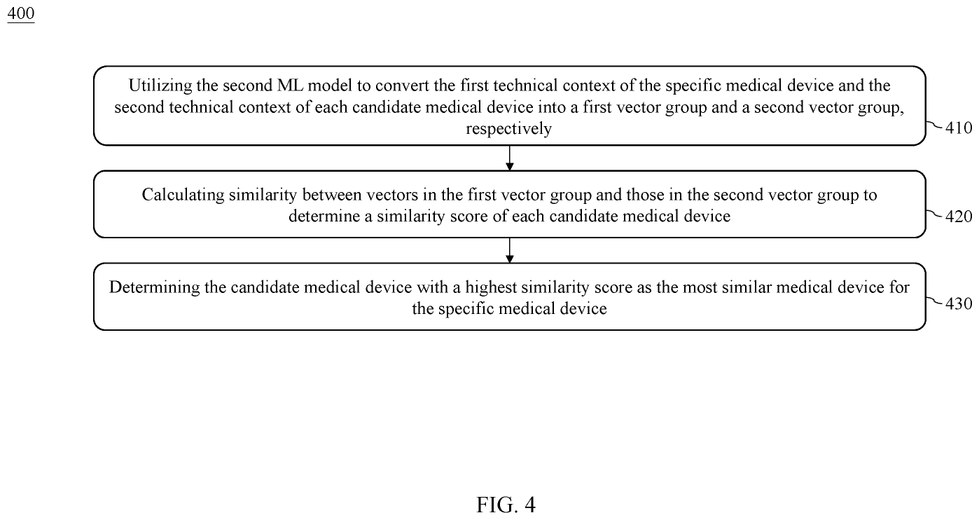

Step 270: Determining a most similar medical device for the specific device according to a similarity score for each candidate medical device calculated from the second technical context of each candidate medical device and the first technical context of the specific medical device using a second machine-learning model. For example, the second machine-learning model may be the machine-learning model 1063. The detailed steps of step 270 will be described in the embodiment of FIG. 4.

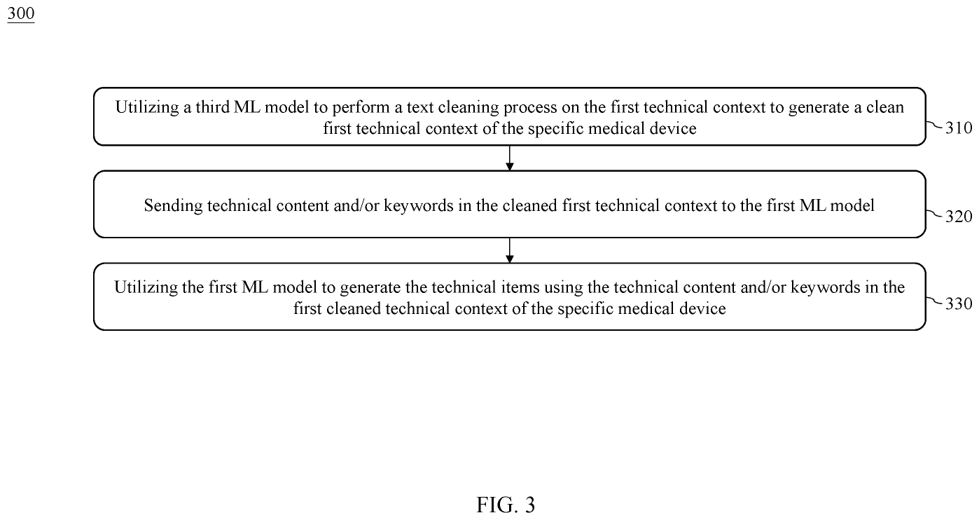

FIG. 3 is a flowchart of step 220 in accordance with the embodiment of in FIG. 2. Please refer to FIG. 2 and FIG. 3.

In an embodiment, step 220 in FIG. 2A may include steps 310 to 330 in the flow 300 shown in FIG. 3. Step 310: Utilizing a third ML model to perform a text cleaning process on the first technical context to generate a cleaned first technical context of the specific medical device. For example, the third machine-learning model may be the machine-learning model 1062. The machine-learning model 1062 used in this process may be a natural language processing (NLP) model that can identify relationships between the various elements of language, such as the letters, words, phrases, and sentences present within in the first technical context. Additionally, the machine-learning model 1062 is capable of identifying and removing adverbs, punctuations, stopwords, and other unwanted elements from the first technical context, thereby facilitating the text cleaning process.

Step 320: Sending technical content and/or keywords in the cleaned first technical context to the first ML model. For example, after the text cleaning process is completed, technical content (e.g., primary technical content) and/or keywords will be retained in the raw text of the cleaned first technical context.

Step 330: Utilizing the first ML model to generate the technical items using the technical content and/or keywords in the cleaned first technical context of the specific medical device. For example, the content and/or keywords within the cleaned first technical context utilized by the remote machine-learning model 30 can yield more precise inferred technical items, accompanied with their respective technical analysis and potential application domains, in comparison to the original first technical context of the specific medical device.

FIG. 4 is a flowchart of step 270 in accordance with the embodiment of in FIG. 2.

Step 410: Utilizing the second ML model to convert the first technical context of the specific medical device and the second technical context of each candidate medical device into a first vector group and a second vector group, respectively. For example, the second machine-learning model may be the machine-learning model 1063, which is a pre-trained natural language processing model capable of converting the specific technical elements in the first technical context of the specific medical device and the abstract of the second technical context of each potential medical device into distinct vector groups using the “word to vector” technique, such as Word2vec.

Step 420: Calculating similarity between vectors in the first vector group and those in the second vector group to determine a similarity score of each candidate medical device. For example, the similarity between the vectors in the first vector group and those in the second vector group may be a cosine similarity, but the present disclosure is not limited thereto.

Step 430: Determining the candidate medical device with a highest similarity score as the most similar medical device for the specific medical device. For example, each candidate medical device on the list may have its own respective similarity score. A higher similarity score of a particular candidate medical device indicates a higher similarity to the specific medical device. In some embodiments, the candidate medical devices with the highest similarity scores (e.g., top 5 highest similarity scores) may be determined as the most similar medical devices for the specific medical device.

The scope of the present disclosure is not intended to be limited to the particular embodiments of the process, machine, manufacture, and composition of matter, means, methods, steps, and operations described in the specification. As those skilled in the art will readily appreciate from the disclosure of the present disclosure, processes, machines, manufacture, composition of matter, means, methods, steps, or operations presently existing or later to be developed that perform substantially the same function or achieve substantially the same result as the corresponding embodiments described herein may be utilized according to the present disclosure. Accordingly, the appended claims are intended to include within their scope processes, machines, manufacture, and compositions of matter, means, methods, steps, or operations. In addition, each claim constitutes a separate embodiment, and the combination of various claims and embodiments are within the scope of the disclosure.

The methods, processes, or operations according to embodiments of the present disclosure can also be implemented on a programmed processor. However, the controllers, flowcharts, and modules may also be implemented on a general purpose or special purpose computer, a programmed microprocessor or microcontroller and peripheral integrated circuit elements, an integrated circuit, a hardware electronic or logic circuit such as a discrete element circuit, a programmable logic device, or the like. In general, any device on which resides a finite state machine capable of implementing the flowcharts shown in the figures may be used to implement the processor functions of the present disclosure.

An alternative embodiment preferably implements the methods, processes, or operations according to embodiments of the present disclosure on a non-transitory, computer-readable storage medium storing computer programmable instructions. The instructions are preferably executed by computer-executable components preferably integrated with a network security system. The non-transitory, computer-readable storage medium may be stored on any suitable computer readable media such as RAMs, ROMs, flash memory, EEPROMs, optical storage devices (CD or DVD), hard drives, floppy drives, or any suitable device. The computer-executable component is preferably a processor, but the instructions may alternatively or additionally be executed by any suitable dedicated hardware device. For example, an embodiment of the present disclosure provides a non-transitory, computer-readable storage medium having computer programmable instructions stored therein.

While the present disclosure has been described with specific embodiments thereof, it is evident that many alternatives, modifications, and variations may be apparent to those skilled in the art. For example, various components of the embodiments may be interchanged, added, or substituted in the other embodiments. Also, all of the elements of each figure are not necessary for operation of the disclosed embodiments. For example, one of ordinary skill in the art of the disclosed embodiments would be able to make and use the teachings of the present disclosure by simply employing the elements of the independent claims. Accordingly, embodiments of the present disclosure as set forth herein are intended to be illustrative, not limiting. Various changes may be made without departing from the spirit and scope of the present disclosure.

Even though numerous characteristics and advantages of the present disclosure have been set forth in the foregoing description, together with details of the structure and function of the invention, the disclosure is illustrative only. Changes may be made to details, especially in matters of shape, size, and arrangement of parts, within the principles of the invention to the full extent indicated by the broad general meaning of the terms in which the appended claims are expressed.

What is claimed is:

1. A method for retrieving information about similar medical devices, the method comprising:
obtaining a first technical context of a specific medical device;
utilizing a first machine-learning model to extract one or more technical items of the specific medical device based on technical content of the first technical context;
utilizing the first machine-learning model to generate one or more candidate medical devices using the one or more technical items of the specific medical device;
searching a database for respective device information about each candidate medical device;
retrieving a summary file of each candidate medical device from the database based on the respective device information about each candidate medical device;
utilizing the first machine-learning model to infer a second technical context of each candidate medical device from the summary file or each candidate medical device; and
determining a most similar medical device for the specific medical device according to a similarity score for each candidate medical device calculated from the second technical context of each candidate medical device and the first technical context of the specific medical device using a second machine-learning model.

2. The method of claim 1, wherein the first technical context comprises an intended use, technical characteristics, technical improvements, and advantages of the specific medical device.

3. The method of claim 1, wherein the first machine-learning model is a large generative pre-trained transformer (GPT) model.

4. The method of claim 1, wherein each technical item comprises a potential application domain and corresponding technical description, and the step of utilizing the first machine-learning model to extract the one or more technical items of the specific medical device based on the technical content of the first technical context comprises:
utilizing a third machine-learning model to perform a text cleaning process on the first technical context of the specific medical device to generate a clean first technical context;
sending the technical content and/or keywords in the cleaned first technical context to the first machine-learning model; and
utilizing the first machine-learning model to generate the technical items using the technical content and/or keywords in the cleaned first technical context of the specific medical device.

5. The method of claim 4, wherein the text cleaning process removes adverbs, punctuations, and stopwords in the first technical context.

6. The method of claim 4, further comprising: determining respective manufacturers of the candidate medical devices based on the potential application domains of the one or more technical items of the specific medical device.

7. The method of claim 1, wherein the database is a public database containing information about predicate medical devices.

8. The method of claim 7, wherein the respective device information about each candidate medical device comprises a device class, a submission number, applicant, device name, and decision date thereof.

9. The method of claim 1, wherein the step of determining the most similar medical device for the specific medical device according to the similarity score for each candidate medical device calculated from the second technical context of each candidate medical device and the first technical context of the specific medical device using the second machine-learning model comprises:
utilizing the second machine-learning model to convert the first technical context of the specific medical device and the second technical context of each candidate medical device into a first vector group and a second vector group, respectively;
calculating similarity between vectors in the first vector group and those in the second vector group to determine the similarity score of each candidate medical device; and
determining the candidate medical device with a highest similarity score as the most similar medical device for the specific medical device.

10. The method of claim 9, wherein the calculated similarity is cosine similarity.

11. A computer device for retrieving information about similar medical devices, the computer device comprising:
a memory having computer executable instructions stored therein; and
a processor coupled to the memory,
wherein the computer executable instructions cause the processor to perform operations,
and the operations comprise:
obtaining a first technical context of a specific medical device;
utilizing a first machine-learning model to extract one or more technical items of the specific medical device based on technical content of the first technical context;
utilizing the first machine-learning model to generate one or more candidate medical devices using the one or more technical items of the specific medical device;
searching a database for respective device information about each candidate medical device;
retrieving a summary file of each candidate medical device from the database based on the respective device information about each candidate medical device;
utilizing the first machine-learning model to infer a second technical context of each candidate medical device from the summary file or each candidate medical device; and
determining a most similar medical device for the specific medical device according to a similarity score for each candidate medical device calculated from the second technical context of each candidate medical device and the first technical context of the specific medical device using a second machine-learning model.

12. The computer device of claim 11, wherein the first technical context comprises an intended use, technical characteristics, technical improvements, and advantages of the specific medical device.

13. The computer device of claim 11, wherein the first machine-learning model is a large generative pre-trained transformer (GPT) model.

14. The computer device of claim 11, wherein each technical item comprises a potential application domain and corresponding technical description, and the step of utilizing the first machine-learning model to extract the one or more technical items of the specific medical device based on the technical content of the first technical context comprises:
utilizing a third machine-learning model to perform a text cleaning process on the first technical context of the specific medical device to generate a clean first technical context;
sending technical content and/or keywords in the cleaned first technical context to the first machine-learning model; and
utilizing the first machine-learning model to generate the technical items using the technical content and/or keywords in the cleaned first technical context of the specific medical device.

15. The computer device of claim 14, wherein the text cleaning process removes adverbs, punctuations, and stopwords in the first technical context.

16. The computer device of claim 14, wherein the operations further comprise: determining respective manufacturers of the candidate medical devices based on the potential application domains of the one or more technical items of the specific medical device.

17. The computer device of claim 11, wherein the database is a public database containing information about predicate medical devices.

18. The computer device of claim 17, wherein the respective device information about each candidate medical device comprises a device class, a submission number, applicant, device name, and decision date of each candidate medical device.

19. The computer device of claim 11, wherein the step of determining the most similar medical device for the specific medical device according to the similarity score for each candidate medical device calculated from the second technical context of each candidate medical device and the first technical context of the specific medical device using the second machine-learning model comprises:
utilizing the second machine-learning model to convert the first technical context of the specific medical device and the second technical context of each candidate medical device into a first vector group and a second vector group, respectively;
calculating similarity between vectors in the first vector group and those in the second vector group to determine the similarity score of each candidate medical device; and
determining the candidate medical device with a highest similarity score as the most similar medical device for the specific medical device.

20. The computer device of claim 19, wherein the calculated similarity is cosine similarity.
